(19) United States
(12) Patent Application Publication
(10) Pub. No.: US 2025/0281027 A1
(43) Pub. Date: Sep. 11, 2025

(54) SUPPORT ASSEMBLY FOR HOLDING A VIDEOSCOPE ON A ROBOTIC SURGICAL SYSTEM

(71) Applicant: EndoQuest Robotics, Inc., Houston, TX (US)

(72) Inventors: Graham Johnson, Houston, TX (US); Yongman Park, Houston, TX (US); Sungwoo Cho, Houston, TX (US); Daniel Kim, Houston, TX (US); Raymond Lee, Houston, TX (US); Dongsuk Shin, Houston, TX (US)

(73) Assignee: EndoQuest Robotics, Inc., Houston, TX (US)

(21) Appl. No.: 18/596,171

(22) Filed: Mar. 5, 2024

(51) Int. Cl. A61B 1/00; A61B 34/30

(52) U.S. Cl. CPC A61B 1/00154; A61B 1/00066; A61B 1/00128; A61B 34/30; A61B 2034/301

(57) ABSTRACT

An assembly for holding a videoscope on a robotic surgical device is disclosed, which includes a holder body having a proximal end portion and a distal end portion, a cradle provided on the proximal end portion of the holder body for supporting a videoscope, a first locking mechanism operatively associated with the cradle for temporarily securing the videoscope in the cradle, a connector provided on the distal end portion of the holder body for attaching the holder body to the robotic surgical device, and a second locking mechanism operatively associated with the connector for temporarily securing the holder body to the robotic surgical device.

### BACKGROUND OF THE DISCLOSURE

### 1. Field of the Invention

The subject disclosure is directed to robotic surgical systems, and more particularly, to a support assembly for temporarily holding a videoscope used with a robotically controlled medical device to enable a surgeon to manually navigate the videoscope to a surgical site under visualization without assistance.

### 2. Description of Related Art

In the performance of robotically assisted transluminal and endoluminal surgical procedures performed in or through a natural orifice of a patient, it is necessary to introduce an elongated videoscope camera to the surgical site. Typically, a surgeon will manually navigate the distal tip of the videoscope to the surgical site under visualization, while an assistant holds the proximal portion or housing of the videoscope.

It would be beneficial however, to enable a surgeon to introduce the videoscope to the surgical site without an assistant holding the videoscope. This would permit the assistant to perform more meaningful tasks during the initial phase of the procedure.

The subject disclosure provides such a beneficial solution by way of a support assembly for temporarily holding a videoscope to enable a surgeon to manually navigate the distal tip of the videoscope to the surgical site under visualization and without assistance.

### SUMMARY OF THE DISCLOSURE

The subject disclosure is directed to a new and useful assembly for holding a videoscope on a robotic surgical device, which includes an elongated holder body having a proximal end portion and a distal end portion. A cradle is provided on the proximal end portion of the holder body for supporting a videoscope and a first locking mechanism is operatively associated with the cradle for temporarily securing the videoscope in the cradle. A connector is provided on the distal end portion of the holder body for attaching the holder body to the robotic surgical device, and a second locking mechanism is operatively associated with the connector for temporarily securing the holder body to the robotic surgical device.

The cradle on the proximal end portion of the holder body includes a first engagement feature adapted to receive a complementary engagement feature on the videoscope, and the connector on the distal end portion of the holder body includes a second engagement feature adapted for reception in a complementary engagement feature on the robotic surgical device. In an embodiment of the subject disclosure, the first engagement feature is an engagement channel formed in the cradle of the holder body and the complementary engagement feature on the videoscope is an elongated engagement flange for engaging the engagement channel in the cradle, and the second engagement feature is an elongated engagement flange on the distal end portion of the holder body and the complementary engagement feature on the robotic surgical device is an engagement channel formed in a connective bracket for receiving the engagement flange of the holder body.

The first locking mechanism includes a cantilevered locking lever and a retention clasp on the cantilevered locking lever for releasably engaging a capture slot in the engagement flange on the videoscope. The second locking mechanism includes a spring-loaded locking slider and a retention tang on the spring-loaded locking slider for releasably engaging a retention groove in the connector of the robotic surgical device. The second locking mechanism further includes a pair of elongated spaced apart retention pins on the spring-loaded locking slider for releasably engaging a pair of corresponding spaced apart retention ports in a proximal end of the robotic surgical device adjacent the connective bracket.

The assembly further includes a hand rest operatively associated with the distal end portion of the holder body for supporting a user's hand. More particularly, the holder body defines a longitudinal axis and the hand rest extends in an axial direction relative to the distal end portion of the holder body and is mounted for pivotal movement between a first position retracted within an internal cavity of the holder body and a second position extending outwardly from the internal cavity of the holder body. A retention clasp is provided on the hand rest for engaging a retention slot in a side wall of the holder body to retain the hand rest in the second position.

The subject disclosure is also directed to a method for holding a videoscope on a robotic surgical device, which includes the steps of providing a holder body having a proximal end portion and a distal end portion; temporarily securing the distal end portion of the holder body to the robotic surgical device; and temporarily securing the videoscope on the proximal end portion of the holder body.

Preferably, the step of temporarily securing the distal end portion of the holder body to the robotic device includes mating an engagement flange on the distal end portion of the holder body with a complementary engagement channel on the robotic surgical device. And the step of temporarily securing the distal end portion of the holder body to the robotic device further includes activating a first locking mechanism that prevents inadvertent disengagement of the engagement flange on the distal end portion of the holder body and the complementary engagement channel on the robotic surgical device.

Preferably, the step of temporarily securing the videoscope on the proximal end portion of the holder body includes mating an engagement flange on the videoscope with a complementary engagement channel on the proximal end portion of the holder body. And the step of temporarily securing the videoscope on the proximal end portion of the holder body further includes activating a second locking mechanism that prevents inadvertent disengagement of the engagement flange on the videoscope and the complementary engagement channel on the proximal end portion of the holder body.

The method further includes the step of moving a hand rest from a first position retracted within an internal cavity of the holder body to a second position extending outwardly from the internal cavity of the holder body. And the method also includes the step of securing the hand rest in the second position by engaging a retention clasp on the hand rest in a retention slot formed in the holder body.

These and other features of the subject disclosure will become more readily apparent to those having ordinary skill in the art to which the subject invention appertains from the detailed description of the preferred embodiments taken in conjunction with the following brief description of the drawings.

### BRIEF DESCRIPTION OF THE DRAWINGS

So that those skilled in the art will readily understand how to make and use the support assembly of the subject disclosure without undue experimentation, preferred embodiments thereof will be described in detail herein below with reference to the figures wherein:

FIG. 1 is an illustration of a surgeon utilizing the support assembly while it is attached to a robotic surgical device to introduce a videoscope into a body lumen of a patient;

FIG. 2 is a side elevational view of the support assembly attached to a robotic surgical device and holding a videoscope with the handle of the support assembly in a deployed position;

FIG. 3 is a side elevational view of the support assembly detached from a robotic surgical device with the videoscope separated from support assembly;

FIG. 4 is an illustration showing engagement of a videoscope within the cradle of the support assembly;

FIG. 5 is a side elevational view of the support assembly with the side wall broken away to illustrate movement of the handle from a stowed position into a deployed position;

FIGS. 6 through 8 illustrate the securement of the engagement flange of the videoscope with the cantilevered locking lever of the support assembly;

FIG. 9 is a perspective view of the support assembly of the subject disclosure;

FIG. 10 is an enlarged perspective view of the distal end portion of the support assembly with a side wall broken away to illustrate the internal features of the spring-loaded locking mechanism for securing the support assembly to a proximal end of the robotic surgical device;

FIG. 11 is another perspective view of the support assembly of the subject disclosure;

FIG. 12 is an illustration showing the complementary engagement features on the distal end of the support assembly and the proximal end of the robotic surgical device;

FIGS. 13 through 15 illustrate the securement of the engagement flange of the videoscope with the spring loaded locking slider of the support assembly; and

FIG. 16 illustrates the videoscope and the robot surgical device engaged with the robotic console for use during a robotically assisted surgical procedure after having been separated from the support assembly after the videoscope has been positioned in the body lumen of the patient.

### DETAILED DESCRIPTION OF THE PREFERRED EMBODIMENTS

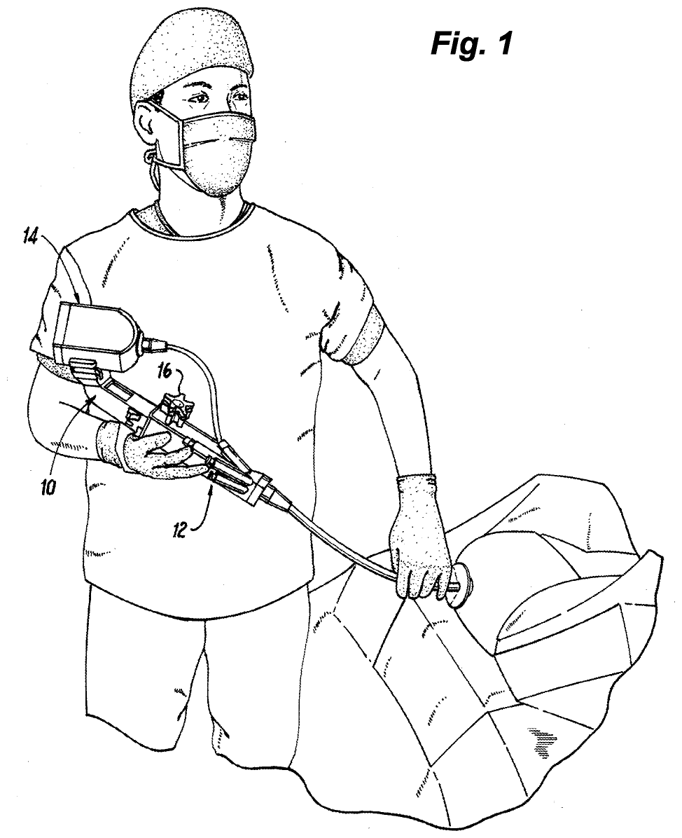

Referring now to the drawings, wherein like reference numerals identify similar structural elements and features of the subject disclosure, there is illustrated in FIG. 1 the support assembly 10 of the subject invention, which is temporarily attached to the proximal end of a robotic surgical device 12. The support assembly 10 is shown holding a videoscope 14 to enable a surgeon to manually navigate the distal end of the videoscope 14 to a surgical site within a body lumen of a patient under visualization, using the manual steering knob 16 of the surgical device 12, and without assistance from operating room staff.

A robotic surgical device of the type illustrated in FIG. 1 is disclosed in commonly assigned U.S. Patent Application Publication No. 2023/0210618, the disclosure of which is herein incorporated by reference in its entirety. The robotic surgical device shown in FIG. 1 is designed for use in conjunction with a patient console of a robotic surgical system of the type disclosed in commonly assigned U.S. Patent Application Publication No. 2023/0285098, the disclosure of which is herein incorporated by reference in its entirety.

As illustrated in FIG. 1, when the support assembly 10 is temporarily attached to the robotic surgical device 12, the elongated flexible camera 18 of videoscope 14 is extended through a working channel 17 of the surgical device 12 and into the steerable shaft 19 of the surgical device 12, which is controlled by the manual steering knob 16 of the surgical device 12.

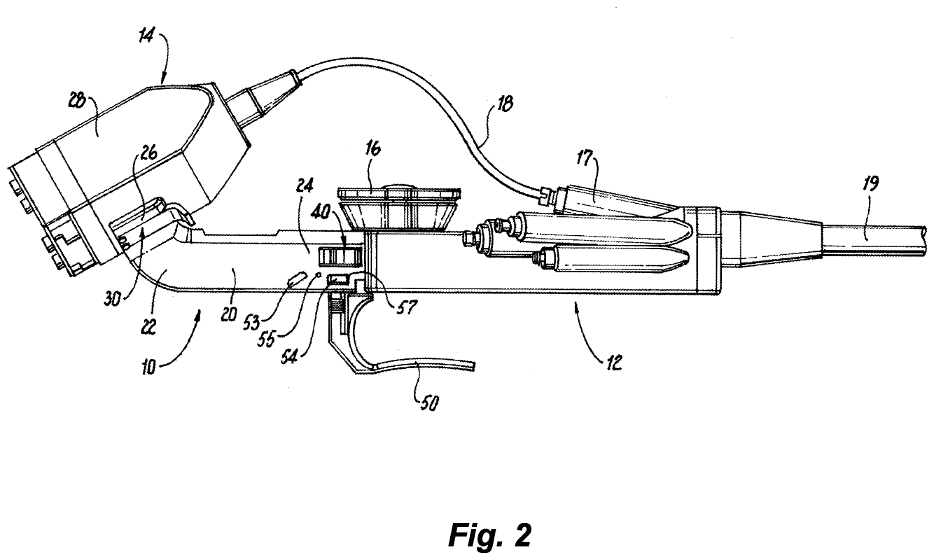

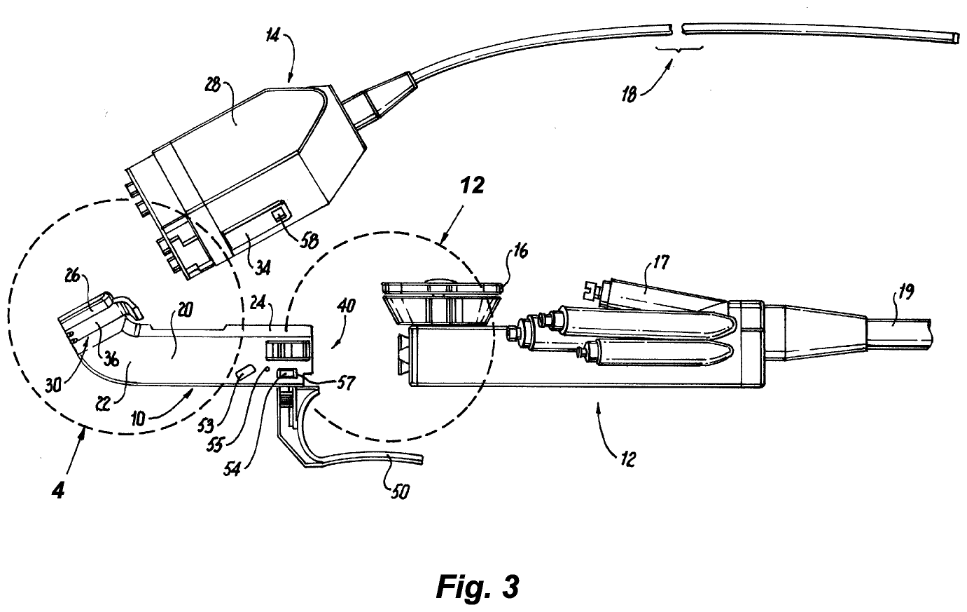

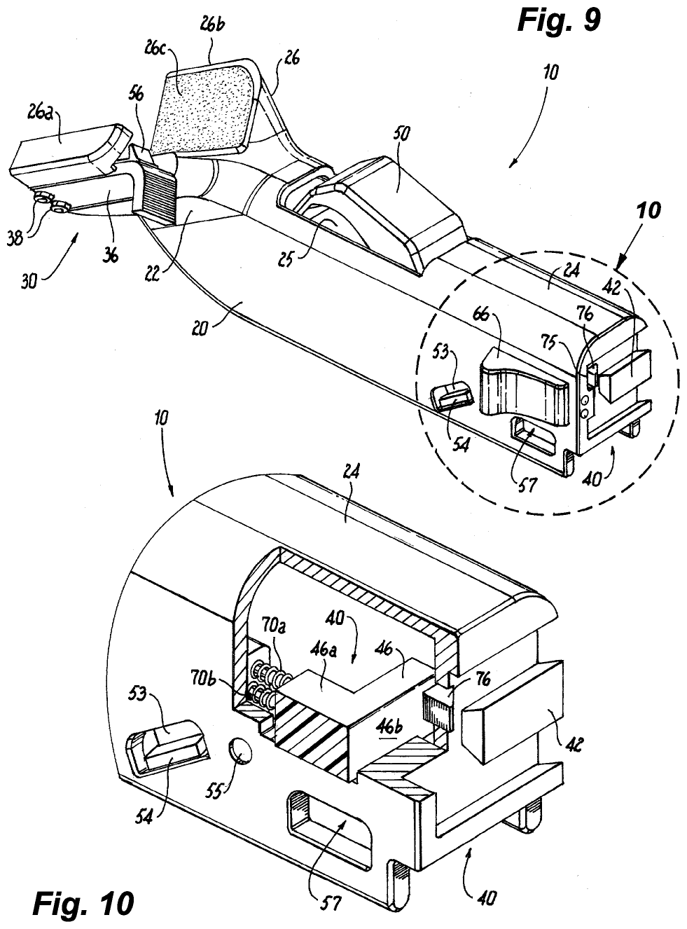

Referring now to FIGS. 2 and 3, the support assembly 10 of the subject disclosure includes an elongated body 20 having opposed proximal and distal end portions 22 and 24. The proximal end portion 22 is curved and upturned from the axis of the body 20. It includes a generally V-shaped cradle 26 for holding the housing 28 of the videoscope 14. The cradle 26 includes a first side arm 26a having locking mechanism 30 for temporality securing the videoscope 14 in the cradle 26 and a second side arm 26b with a frictional retention pad 26c positioned therein, which is best seen in FIG. 9.

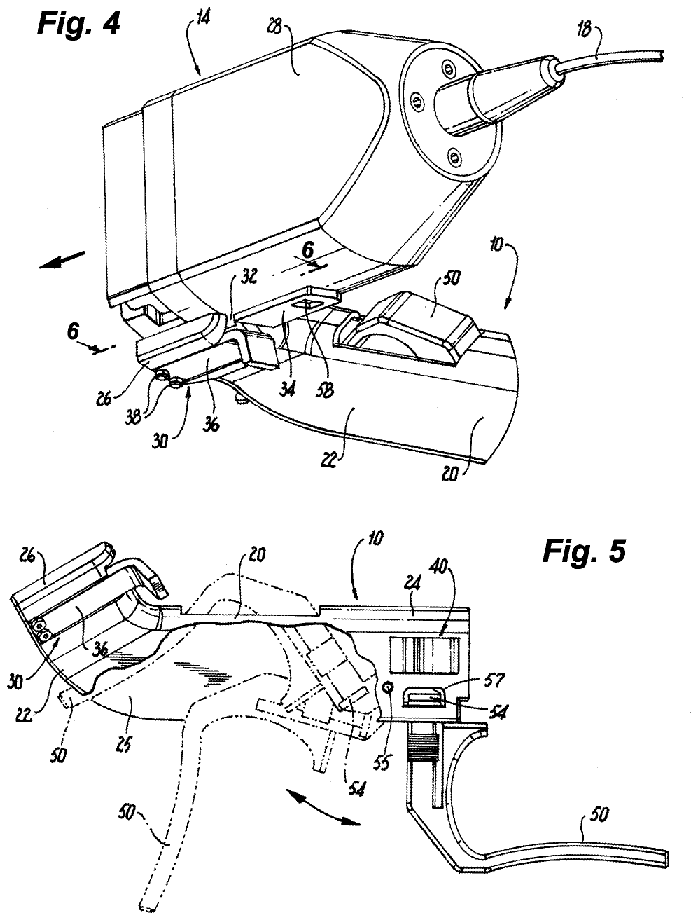

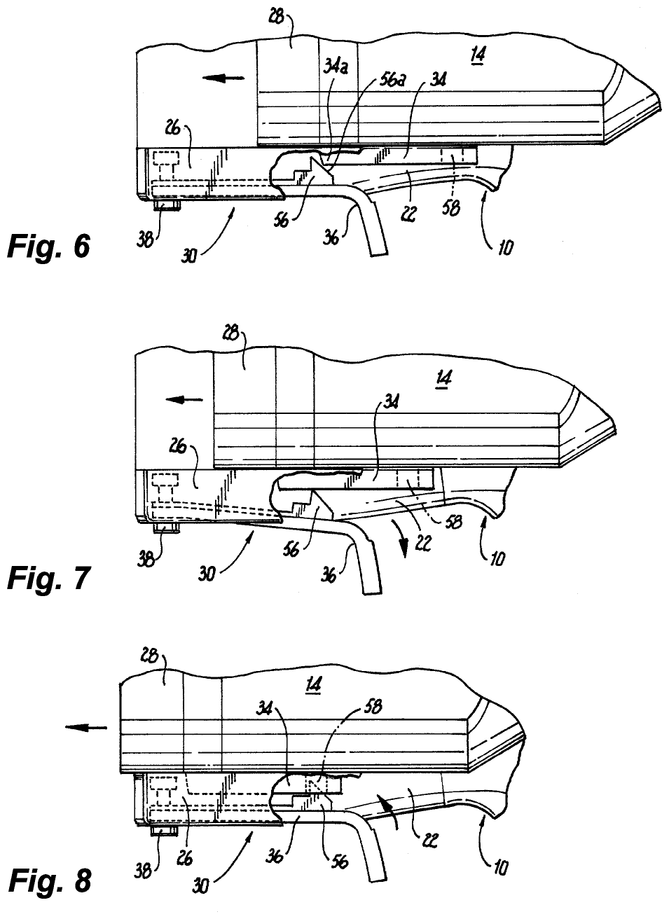

Locking mechanism 30 includes a first engagement feature located within the cradle 26 in the form of an elongated engagement channel 32 and a second engagement feature on the housing 28 of videoscope 14 in the form of a complementary rectangular engagement flange 34, which are best seen in FIG. 4. The locking mechanism 30 further includes a generally L-shaped cantilevered locking lever 36 fixed at one end by fasteners 38 for temporarily securing the engagement flange 34 with the engagement slot 32. The structure and function of locking mechanism 30 will be described in more detail below with reference to FIGS. 6 through 8.

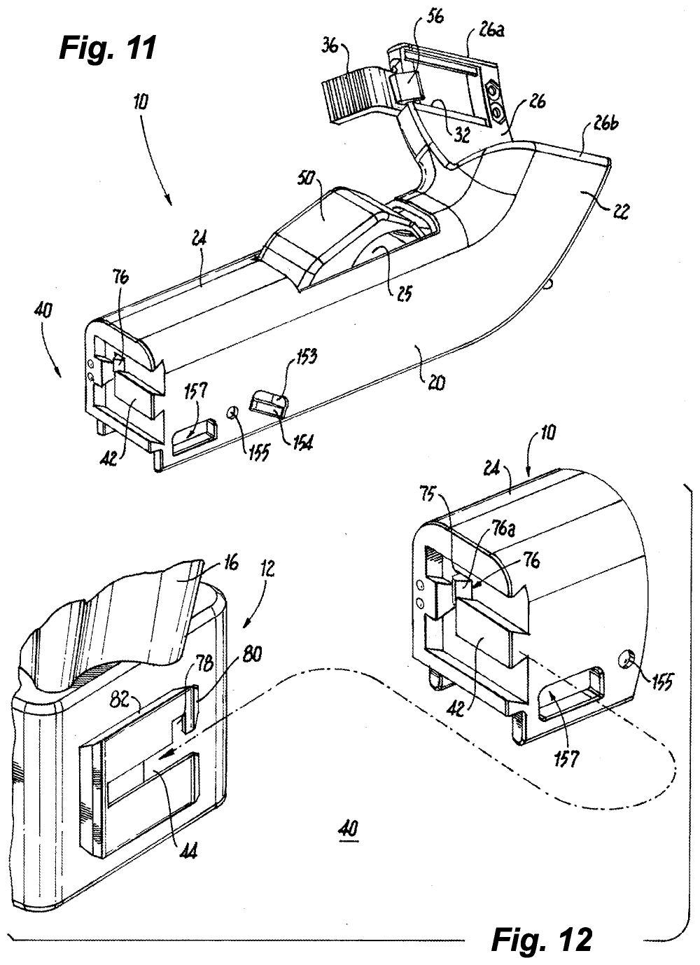

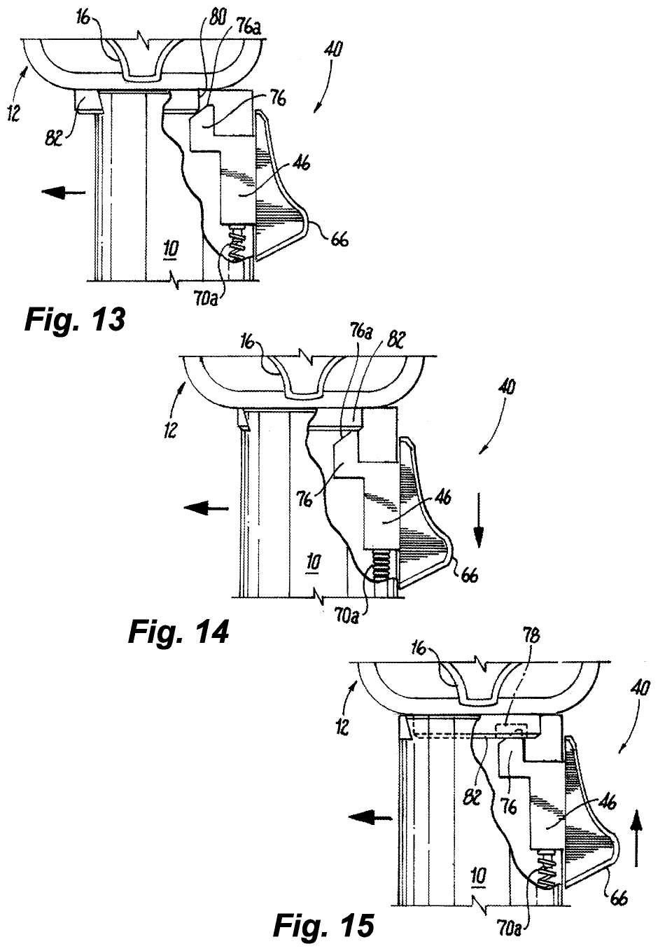

With continuing reference to FIGS. 2 and 3, the distal end portion 24 of the body 20 of support assembly 10 incudes a second locking mechanism 40 for temporality securing the body 20 to the proximal end of the robotic surgical device 12. Locking mechanism 40 includes, among other features, a first engagement feature located on the distal end portion 24 of body 20 in the form of a dovetail-shaped engagement flange 42 and a second engagement feature on the proximal end of the robotic surgical device 12 in the form of a complementary engagement channel 44 formed in a connective bracket 82, as best seen in FIGS. 11 and 12. The locking mechanism 40 further includes a spring-loaded locking slider 46 for temporarily securing the engagement flange 44 within the engagement slot 44, which is best seen in FIGS. 9 and 10. The structure and function of locking mechanism 40 will be described in more detail below with reference to FIGS. 13 through 15.

Referring now to FIG. 5, the support assembly 10 of the subject disclosure further includes a deployable generally J-shaped hand rest 50 operatively associated with the distal end portion 24 of the holder body 20 for supporting a surgeon's hand during the manual navigation of the distal end of the videoscope 14 to a surgical site. The hand rest 50 extends in an axial direction relative to the distal end portion 24 of the holder body 20 and is mounted for pivotal movement between a first position retracted within an internal cavity 25 of the holder body 20 and a second position extending outwardly and downwardly from the internal cavity 25 of the holder body 20. In the retracted position, the hand rest 50 is secured from movement through the engagement of retention clasp 54 in a first generally rectangular slot 53 formed in the side wall of the body 20.

The hand rest 50 rotates about a pivot pin 55 and is secured in the deployed downward position through the engagement of the retention clasp 54 on the upper end of the hand rest 50 within a second generally rectangular retention slot 57 formed in the side wall of the body 20. The same hand rest pivot and retention features are shown on the opposite side wall of the support assembly in FIGS. 11 and 12, and identified as pivot pin 155, retention clasp 154, and first and second retention slots 153 and 157.

Referring now to FIGS. 6 through 8, there is illustrated the way the components of locking mechanism 30 function to temporarily secure the videoscope 14 in the cradle 26 of support assembly 10. Initially, the rectangular engagement flange 34 on the housing 28 of videoscope 14 is aligned with the engagement channel 32 in the cradle 26 of the support assembly 10. Then, the videoscope 14 is moved in a proximal direction so that the engagement flange 34 slides proximally within the engagement channel 32. Eventually, the sloped leading edge 34a of engagement flange 34 meets the sloped front camming face 56a of a retention clasp 56 that projects upwardly from the top surface of lever 36, as shown in FIG. 6.

As the flange 34 continues to move proximally in channel 32, the front face 56a of retention clasp 56 is cammed or otherwise urged in a downward direction until it runs along the undersurface of the engagement flange 34, as best seen in FIG. 7. Continued movement of the videoscope 14 in a proximal direction enables the retention clasp 56 to flex back in an upward direction to engage the capture slot 58 formed in the distal end portion of engagement flange 34, as shown in FIG. 8. This mechanical engagement releasably secures the position of the videoscope 14 within the cradle 26 of support assembly 10. Moreover, it prevents the inadvertent disengagement of the videoscope 14 from the cradle 26. At an appropriate time, the retention clasp 56 can be readily released from the capture slot 58 by pulling the lever 36 downward. Then, the videoscope 14 can be moved distally to disengage flange 34 from channel 32.

Referring once again to FIGS. 9 through 12, as mentioned above, the second locking mechanism 40 for temporality securing the body 20 to the proximal end of the robotic surgical device 12 includes a spring-loaded locking slider 46 for temporarily securing the engagement flange 44 within the engagement slot 44. Slider 46 is associated with a sliding switch 66 configured for manual movement by a user. A pair of axially retained and parallel arranged compression springs 70a and 70b are operatively associated with the proximal surface of the shorter leg 46a of slider 46 for biasing the slider 46 in a distal direction, which corresponds to a locked position of the mechanism 40.

Locking mechanism 40 further includes an engagement tang 76 which extends proximally from the longer leg 46b of slide 46, and through a slot 75 in the proximal end 24 of support structure 10, for releasably engaging a corresponding retention groove 78 formed within the engagement slot 44 at the proximal end of robotic surgical device 12, as best seen in FIG. 12.

Referring now to FIGS. 13 through 15, there is illustrated the way the components of locking mechanism 40 function to temporarily secure the distal end portion 24 of the support assembly 10 to the proximal end of the robotic surgical device 12. Initially, the dovetail shaped engagement flange 42 on the proximal end portion 24 of support assembly 10 is aligned with the engagement channel 44 at the proximal end of surgical device 12 as illustrated in FIG. 12. Then, the support assembly 10 is moved in a lateral direction so that the engagement flange 42 slides into the engagement channel 44. Eventually, the leading edge 80 of the connective bracket 82 that forms channel 44 meets the sloped front face 76a of the engagement tang 76 that projects proximally from slide block 46, as shown in FIG. 13.

Continued lateral movement of the support structure 10 enables the slide block 46 to cammed or otherwise move proximally against the bias of springs 70a, 70b, as shown in FIG. 14. Eventually, the engagement tang 76 will align with retention groove 78 and the slide block 46 will translate distally causing the engagement tang 76 to releasably engage groove 78. This mechanical engagement releasably secures the support assembly 10 to the proximal end of robotic surgical device 12. Moreover, it prevents the inadvertent disengagement of the support assembly 10 from the robotic surgical device 12. At an appropriate time, the surgeon can slide switch 66 in a proximal direction to release the tang 76 from groove 78, and the engagement flange 42 can be slid laterally out of the slot 44 formed in connective bracket 82.

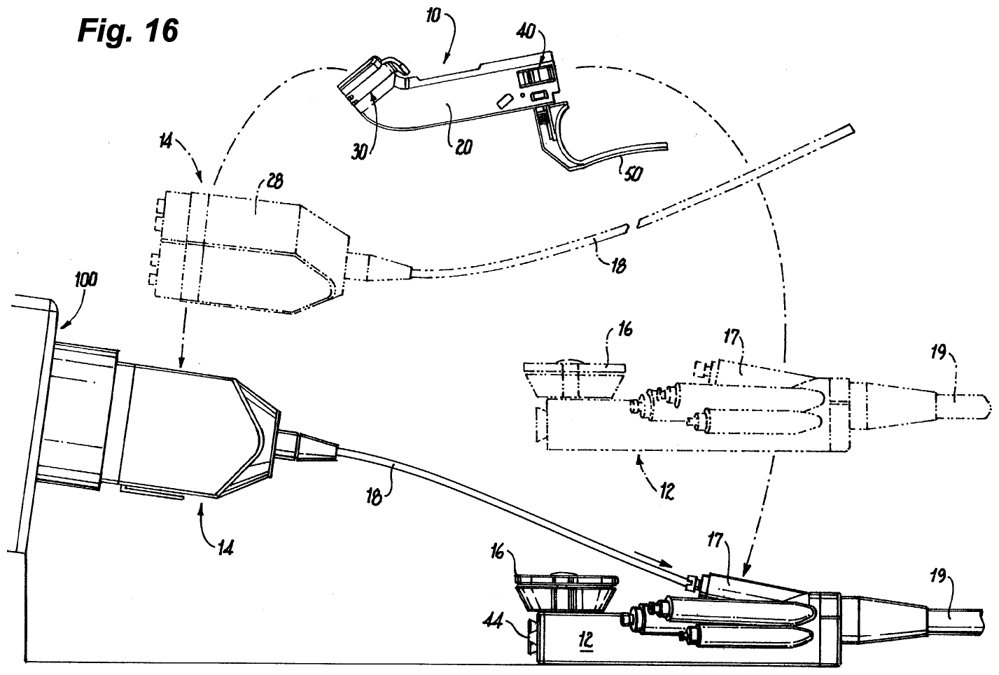

As illustrated in FIG. 16, when the distal end of the flexible camera 18 has reached the surgical site within the body lumen of the patient, having been navigated there by a surgeon under visualization and without assistance, the distal end portion 24 of support assembly 10 can be released or otherwise disengaged from the proximal end of the robotic surgical device 12, and the videoscope 14 can be released from the cradle 26 of support assembly 10. Thereafter, the housing 28 of videoscope 14 can be attached to the robotic surgical system 100 for use during the performance of a surgical procedure, and the support assembly 10 can be stored away for subsequent use.

While the subject disclosure has been shown and described with reference to preferred embodiments, those skilled in the art will readily appreciate that changes and/or modifications may be made thereto without departing from the scope of the subject disclosure.

What is claimed is:

1. A support assembly for holding a videoscope on a robotic surgical device, comprising:
a) a holder body having a proximal end portion and a distal end portion;
b) a cradle provided on the proximal end portion of the holder body for supporting a videoscope;
c) a first locking mechanism operatively associated with the cradle for temporarily securing the videoscope in the cradle;
d) a connector provided on the distal end portion of the holder body for attaching the holder body to the robotic surgical device; and
e) a second locking mechanism operatively associated with the connector for temporarily securing the holder body to the robotic surgical device.

2. The assembly of claim 1, wherein the cradle on the proximal end portion of the holder body includes a first engagement feature adapted to receive a complementary engagement feature on the videoscope.

3. The assembly of claim 1, wherein the connector on the distal end portion of the holder body includes a second engagement feature adapted for reception in a complementary engagement feature on the robotic surgical device.

4. The assembly of claim 2, wherein the first engagement feature is an engagement channel formed in the cradle of the holder body and the complementary engagement feature on the videoscope is an elongated engagement flange for engaging the engagement channel in the cradle.

5. The assembly of claim 3, wherein the second engagement feature is an elongated engagement flange on the distal end portion of the holder body and the complementary engagement feature on the robotic surgical device is an engagement channel formed in a connective bracket for receiving the engagement flange of the holder body.

6. The assembly of claim 4, wherein the first locking mechanism includes a cantilevered locking lever.

7. The assembly of claim 6, wherein the first locking mechanism further includes a retention clasp on the cantilevered locking lever for releasably engaging a capture slot in the engagement flange on the videoscope.

8. The assembly of claim 5, wherein the second locking mechanism includes a spring-loaded locking slider.

9. The assembly of claim 8, wherein the second locking mechanism further includes a retention tang on the spring-loaded locking slider for releasably engaging a retention groove in the connector of the robotic surgical device.

10. The assembly of claim 8, wherein the second locking mechanism further includes a pair of elongated spaced apart retention pins on the spring-loaded locking slider for releasably engaging a pair of corresponding spaced apart retention ports in a proximal end of the robotic surgical device adjacent the connective bracket.

11. The assembly of claim 1, further comprising a hand rest operatively associated with the distal end portion of the holder body for supporting a user's hand.

12. The assembly of claim 11, wherein the holder body defines a longitudinal axis and the hand rest extends in an axial direction relative to the distal end portion of the holder body and is mounted for pivotal movement between a first position retracted within an internal cavity of the holder body and a second position extending outwardly from the internal cavity of the holder body.

13. The assembly of claim 12, further comprising a retention clasp provided on the hand rest for engaging a retention slot in a side wall of the holder body to retain the hand rest in the second position.

14. A method for holding a videoscope on a robotic surgical device, comprising the steps of:
a) providing a holder body having a proximal end portion and a distal end portion;
b) temporarily securing the distal end portion of the holder body to the robotic surgical device; and
c) temporarily securing the videoscope on the proximal end portion of the holder body.

15. The method of claim 14, wherein the step of temporarily securing the distal end portion of the holder body to the robotic device includes mating an engagement flange on the distal end portion of the holder body with a complementary engagement channel on the robotic surgical device.

16. The method of claim 15, wherein the step of temporarily securing the distal end portion of the holder body to the robotic device further includes activating a first locking mechanism that prevents inadvertent disengagement of the engagement flange on the distal end portion of the holder body and the complementary engagement channel on the robotic surgical device.

17. The method of claim 14, wherein the step of temporarily securing the videoscope on the proximal end portion of the holder body includes mating an engagement flange on the videoscope with a complementary engagement channel on the proximal end portion of the holder body.

18. The method of claim 17, wherein the step of temporarily securing the videoscope on the proximal end portion of the holder body further includes activating a second locking mechanism that prevents inadvertent disengagement of the engagement flange on the videoscope and the complementary engagement channel on the proximal end portion of the holder body.

19. The method of claim 14, further comprising the step of moving a hand rest from a first position retracted within an internal cavity of the holder body to a second position extending outwardly from the internal cavity of the holder body.

20. The method of claim 19, further comprising the step of securing the hand rest in the second position by engaging a retention clasp on the hand rest in a retention slot formed in the holder body.
